(19) United States
(12) Patent Application Publication
(10) Pub. No.: US 2026/0290494 A1
(43) Pub. Date: Sep. 24, 2026

(54) METHOD AND APPARATUS FOR PREDICTING ENZYME COMMISSION NUMBER USING AMINO ACID SEQUENCE

(71) Applicant: Jiwoo SIM, Bloomfield Hills, MI (US)

(72) Inventor: Jiwoo SIM, Bloomfield Hills, MI (US)

(21) Appl. No.: 19/082,260

(22) Filed: Mar. 18, 2025

(51) Int. Cl. G16B 15/10; G06N 20/00; G16B 40/20

(52) U.S. Cl. CPC G16B 15/10; G06N 20/00; G16B 40/20

(57) ABSTRACT

Provided are apparatuses, a non-transitory computer-readable medium or media, for predicting an enzyme commission number using an amino acid sequence of a subject. In certain aspects, disclosed a method including the steps of: preprocessing including converting the amino acid sequence into a 2-dimensional embedding matrix having M rows and N columns, creating newly a plurality of the 2-dimensional embedding matrices by shifting at least one row of M rows in a column direction of the 2-dimensional embedding matrix, and transforming the plurality of the 2-dimensional embedding matrices into a 3-dimensional embedding matrix by stacking the plurality of the 2-dimensional embedding matrices together; extracting a feature of the amino acid sequence from the 3-dimensional embedding matrix using a machine learning model; and predicting the enzyme commission number based on the extracted feature of the amino acid sequence.

### A. TECHNICAL FIELD

The present disclosure relates to prediction of an enzyme commission number using an amino acid sequence, more particularly, to an apparatus and method for predicting the enzyme commission number from an extracted feature of the amino acid sequence based on a machine learning model.

### B. DESCRIPTION OF THE RELATED ART

Natural medication, or medicine derived from natural products such as mammalians, bacteria, fungi, and plants, has been utilized by people for millennials. Notably, willow bark extract has been used as an anti-inflammatory and pain reliever for thousands of years (Shara and Stohs 2015). Such plant-based botanical drugs have played an invaluable role in the fields of drug discovery and medication, developing drugs that save the lives of countless people to this day such as Aspirin, Tamiflu, and Paclitaxel. Many botanical drugs have been developed through the existing knowledge of the medical capacities of certain plants that have been used for prolonged periods throughout history. For example, artemisinin was discovered by searching through ancient Chinese herb recipes and discovering the potent antimalarial substance in sweet wormwood.

This led to a revolutionary change in the combat against malaria, eventually being acknowledged with a Nobel Prize in medicine in 2015 (Su and Miller 2015). By already having qualitative data on these plants over decades if not centuries, many plant-based drugs have high chances of success and approval in earlier stages of clinical trials. However, the rate of success in finding a plant and synthesizing a drug is extremely low and also requires considerable amounts of time and resources in testing (Patridge et al. 2016). Thus, the percentage of plant-based New Molecular Entity (NME) products has dropped by more than 50% since 1950, now barely lingering under 8.7% of all NME products (Patridge et al. 2016). Requiring extensive prior knowledge of a plant in order to develop a drug further has been a major challenge, stunting the development of botanical drugs over the years. There are almost 400,000 plant species in the world, with only 31,000 having a documented use, roughly having a usage rate below 8%. Plant-based drugs are likely to have fewer adverse effects and are much more environmentally sustainable than synthetic drugs, making them an incredible asset in the field of medicine.

To expedite the process of plant-based drug discovery and development, it is important to further analyze the properties of potential plants for drug discovery, as it is clear that there is a lack of knowledge and usage of plants that are still open for examination. To do so, it needs to utilize a machine learning based high-throughput screening of the molecular structures of the phytochemicals of plants and further analyze the enzyme commission number of the proteins in the plants to offer a deeper understanding of potential botanical medicine.

### SUMMARY OF THE DISCLOSURE

In one aspect of the present disclosure, apparatus for predicting an enzyme commission number using an amino acid sequence of a subject comprises a processor; and a memory comprising one or more sequences of instructions which, when executed by the processor, causes steps to be performed including: preprocessing including converting the amino acid sequence into a 2-dimensional embedding matrix having M rows and N columns, creating newly a plurality of the 2-dimensional embedding matrices by shifting at least one row of M rows in a column direction of the 2-dimensional embedding matrix, and transforming the plurality of the 2-dimensional embedding matrices into a 3-dimensional embedding matrix by stacking the plurality of the 2-dimensional embedding matrices together; extracting a feature of the amino acid sequence from the 3-dimensional embedding matrix using a machine learning model; and predicting the enzyme commission number based on the extracted feature of the amino acid sequence.

Desirably, the plurality of the 2-dimensional embedding matrix may be created by sequentially shifting one row of the M rows in the column direction.

Desirably, the plurality of the 2-dimensional embedding matrix may be created by sequentially shifting an even row of the M rows in the column direction.

Desirably, the plurality of the 2-dimensional embedding matrix may be created by sequentially shifting an odd row of the M rows in the column direction.

Desirably, the machine learning model may include convolutional neural network or Long Short-Term Model.

### BRIEF DESCRIPTION OF THE DRAWINGS

References will be made to embodiments of the disclosure, examples of which may be illustrated in the accompanying figures. These figures are intended to be illustrative, not limiting. Although the disclosure is generally described in the context of these embodiments, it should be understood that it is not intended to limit the scope of the disclosure to these particular embodiments.

FIG. 1 is a schematic diagram of an illustrative apparatus for predicting an enzyme commission number using an amino acid sequence according to embodiments of the present disclosure.

FIG. 2 is a schematic diagram of an illustrative system for predicting an enzyme commission number using an amino acid sequence according to embodiments of the present disclosure.

FIG. 3 is a view showing an illustrative process for preprocessing an amino acid sequence by an apparatus according to embodiments of the present disclosure.

FIGS. 4A to 4C are an exemplary diagram showing a transformed 3-dimensional embedding matrix after various shifting of an initial 2-dimensional embedding matrix according to embodiments of the present disclosure.

FIG. 5 is an exemplary diagram of an architecture of a machine learning model for learning a process according to embodiments of the present disclosure.

FIG. 6 shows a table of performance comparison of enzyme commission number prediction results achieved by the computing device shown in FIG. 1 based on comparison with 3-dimensional embedding matrix created by another method according to embodiments of the present disclosure.

FIG. 7 is an exemplary flow diagram showing a predicting process of an enzyme commission number according to embodiments of present disclosure.

### DETAILED DESCRIPTION OF PREFERRED EMBODIMENTS

In the following description, for purposes of explanation, specific details are set forth in order to provide an understanding of the disclosure. It will be apparent, however, to one skilled in the art that the disclosure can be practiced without these details. Furthermore, one skilled in the art will recognize that embodiments of the present disclosure, described below, may be implemented in a variety of ways, such as a process, an apparatus, a system, a device, or a method on a tangible computer-readable medium.

Components shown in diagrams are illustrative of exemplary embodiments of the disclosure and are meant to avoid obscuring the disclosure. It shall also be understood that throughout this discussion that components may be described as separate functional units, which may comprise sub-units, but those skilled in the art will recognize that various components, or portions thereof, may be divided into separate components or may be integrated together, including integrated within a single system or component. It should be noted that functions or operations discussed herein may be implemented as components that may be implemented in software, hardware, or a combination thereof.

It shall also be noted that the terms “coupled,” “connected,” “linked,” or “communicatively coupled” shall be understood to include direct connections, indirect connections through one or more intermediary devices, and wireless connections.

Furthermore, one skilled in the art shall recognize: (1) that certain steps may optionally be performed; (2) that steps may not be limited to the specific order set forth herein; and (3) that certain steps may be performed in different orders, including being done contemporaneously.

Reference in the specification to “one embodiment,” “preferred embodiment,” “an embodiment,” or “embodiments” means that a particular feature, structure, characteristic, or function described in connection with the embodiment is included in at least one embodiment of the disclosure and may be in more than one embodiment. The appearances of the phrases “in one embodiment,” “in an embodiment,” or “in embodiments” in various places in the specification are not necessarily all referring to the same embodiment or embodiments.

In the following description, it shall also be noted that the terms “learning” shall be understood not to intend mental action such as human educational activity because of referring to performing machine learning by a processing module such as a processor, a CPU, an application processor, micro-controller, so on.

An “image” is defined as a reproduction or imitation of the form of a person or thing, or specific characteristics thereof, in digital form. An image can be, but is not limited to, a JPEG image, a PNG image, a GIF image, a TIFF image, or any other digital image format known in the art. “Image” is used interchangeably with “photograph”.

A “feature(s)” or “feature information” is defined as a group of one or more descriptive characteristics of subjects that can discriminate for disease. A feature can be a numeric attribute.

The terms “comprise/include” used throughout the description and the claims and modifications thereof are not intended to exclude other technical features, additions, components, or operations.

The terminology used herein is for purposes of describing particular embodiments only and is not intended to be limiting. The defined terms are in addition to the technical and scientific meanings of the defined terms as commonly understood and accepted in the technical field of the present teachings.

Relative terms may be used to describe the various elements' relationships to one another, as illustrated in the accompanying drawings. These relative terms are intended to encompass different orientations of the device and/or elements in addition to the orientation depicted in the drawings.

Unless the context clearly indicates otherwise, the singular forms “a,” “an,” and “the” are intended to include the plural forms as well. Also, when description related to a known configuration or function is deemed to render the present disclosure ambiguous, the corresponding description is omitted.

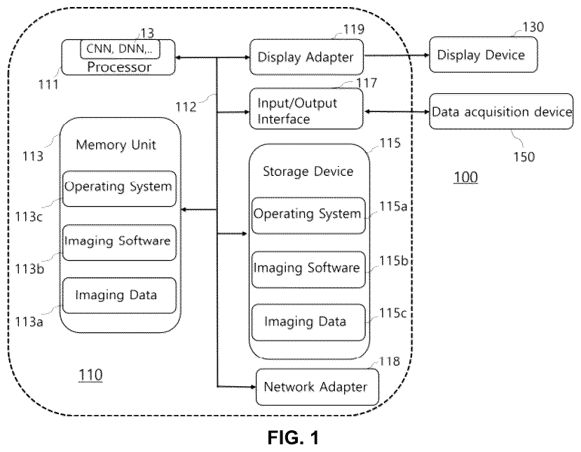

FIG. 1 is a schematic diagram of an illustrative apparatus 100 for predicting an enzyme commission number using an amino acid sequence according to embodiments of the present disclosure.

As depicted, the apparatus 100 may include a computing device 110, a display device 130 and a data acquisition device 150. In embodiments, the computing device 110 may include, but is not limited thereto, one or more processor 111, a memory unit 113, a storage device 115, an input/output interface 117, a network adapter 118, a display adapter 119, and a system bus 112 connecting various system components to the memory unit 113. In embodiments, the apparatus 100 may further include communication mechanisms as well as the system bus 112 for transferring information. In embodiments, the communication mechanisms or the system bus 112 may interconnect the processor 111, a computer-readable medium, a short range communication module (e.g., a Bluetooth, a NFC), the network adapter 118 including a network interface or mobile communication module, the display device 130 (e.g., a CRT, a LCD, etc.), an input device (e.g., a keyboard, a keypad, a virtual keyboard, a mouse, a trackball, a stylus, a touch sensing means, etc.) and/or subsystems. In embodiments, the data acquisition device 150 may be a device that can acquire an amino acid sequence in plants or foods. The acquired data may be stored in the memory unit 113 or the storage device 115, or may be provided to the processor 111 through the input/output interface 117 and processed based on the machine learning model 13.

In embodiments, the processor 111 is configured to perform one or more machine learning models 13, which can be implemented in hardware, software, firmware, or a combination thereof. The processor 111 may be, but is not limited to, a processing module, a Computer Processing Unit (CPU), an Application Processor (AP), a microcontroller, a digital signal processor. In addition, in embodiments, the processor 111 may communicate with a hardware controller such as the display adapter 119 to display a user interface on the display device 130. In embodiments, the processor 111 may access the memory unit 113 and execute commands stored in the memory unit 113 or one or more sequences of instructions to control the operation of the apparatus 100. The commands or sequences of instructions may be read in the memory unit 113 from computer-readable medium or media such as a static storage or a disk drive, but is not limited thereto. In alternative embodiments, a hard-wired circuitry which is equipped with a hardware in combination with software commands may be used. The hard-wired circuitry can replace the soft commands. The instructions may be an arbitrary medium for providing the commands to the processor 111 and may be loaded into the memory unit 113.

In embodiments, the system bus 112 may represent one or more of several possible types of bus structures, including a memory bus or memory controller, a peripheral bus, an accelerated graphics port, and a processor or local bus using any of a variety of bus architectures. For instance, such architectures can comprise an Industry Standard Architecture (ISA) bus, a Micro Channel Architecture (MCA) bus, an Enhanced ISA (EISA) bus, a Video Electronics Standards Association (VESA) local bus, an Accelerated Graphics Port (AGP) bus, and a Peripheral Component Interconnects(PCI), a PCI-Express bus, a Personal Computer Memory Card Industry Association (PCMCIA), Universal Serial Bus (USB) and the like. In embodiments, the system bus 112, and all buses specified in this description can also be implemented over a wired or wireless network connection.

A transmission media including wires of the system bus 112 may include at least one of coaxial cables, copper wires, and optical fibers. For instance, the transmission media may take a form of sound waves or light waves generated during radio wave communication or infrared data communication.

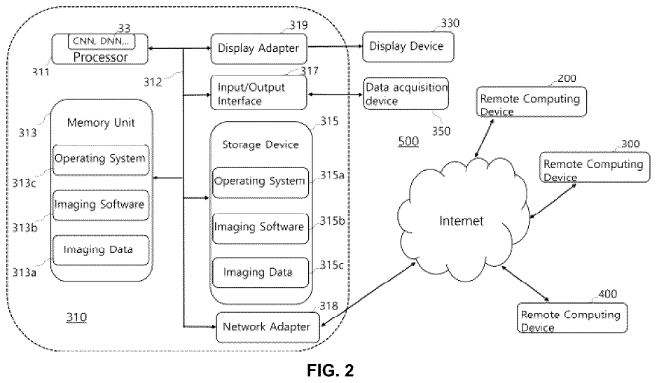

In embodiments, the apparatus 100 may transmit or receive the commands including messages, data, and one or more programs, i.e., a program code, through a network link or the network adapter 118. In embodiments, the network adapter 118 may include a separate or integrated antenna for enabling transmission and reception through the network link. The network adapter 118 may access a network and communicate with a remote computing devices 200, 300, 400 in FIG. 2.

In embodiments, the network may be, but is not limited to, at least one of LAN, WLAN, PSTN, and cellular phone networks. The network adapter 118 may include at least one of a network interface and a mobile communication module for accessing the network. In embodiments, the mobile communication module may be accessed to a mobile communication network for each generation such as 2G to 5G mobile communication network.

In embodiments, on receiving a program code, the program code may be executed by the processor 111 and may be stored in a disk drive of the memory unit 113 or in a non-volatile memory of a different type from the disk drive for executing the program code.

In embodiments, the computing device 110 may include a variety of computer-readable medium or media. The computer-readable medium or media may be any available medium or media that are accessible by the computing device 100. For example, the computer-readable medium or media may include, but is not limited to, both volatile and non-volatile media, removable or non-removable media.

In embodiments, the memory unit 113 may typically stores the amino acid sequence data that are used by the machine learning model 13, as described below in detail, although the database could be at some other location that is external to the remote computing devices 200, 300, 400 and accessible by the processor 111 via a network. The memory unit 113 may store a driver, an application program, data, and a database for operating the apparatus 100 therein. In addition, the memory unit 113 may include a computer-readable medium in a form of a volatile memory such as a random access memory (RAM), a non-volatile memory such as a read only memory (ROM), and a flash memory. For instance, it may be, but is not limited to, a hard disk drive, a solid state drive, an optical disk drive.

In embodiments, each of the memory unit 113 and the storage device 115 may be program modules such as the imaging software 113b, 115b and the operating systems 113c, 115c that can be immediately accessed so that a data such as the imaging data 113a, 115a is operated by the processor 111.

In embodiments, the machine learning model 13 may be trained to classify specific features contained in an amino acid sequence and to predict, based on the classification, an enzyme commission number of the amino acid sequence in a plant subject. The manner in which training may be performed and the manner in which the apparatus 100 is used to predict the enzyme commission number of the amino acid sequence in the plant subject are described below. Once trained, the machine learning model 13 may analyze the amino acid sequence received by the data acquisition device 150 to identify and classify features contained in the amino acid sequence. Based on the classification of the features, the machine learning model 13 may predicts the enzyme commission number of the amino acid sequence. In embodiments, the machine learning model 13 may be installed into at least one of the processors 111, the memory unit 113 and the storage device 115. The machine learning model 13 may use, but is not limited to, at least one of a deep neural network (DNN), a convolutional neural network (CNN) and a recurrent neural network (RNN), which are one of the machine learning algorithms.

FIG. 2 is a schematic diagram of an illustrative system 500 for predicting an enzyme commission number using an amino acid sequence according to embodiments of the present disclosure.

As depicted, the system 500 may include a computing device 310 and one and more remote computing devices 200, 300, 400. In embodiments, the computing device 310 and the remote computing devices 200, 300, 400 may be connected to each other through a network. The components 310, 311, 312, 313, 315, 317, 318, 319, 330 of the system 500 are similar to their counterparts in FIG. 1. In embodiments, each of remote computing devices 200, 300, 400 may be similar to the apparatus 100 in FIG. 1. For instance, each of remote computing devices 200, 300, 400 may include each of the subsystems, including the processor 311, the memory unit 313, an operating system 313c, 315a, an imaging software 313b, 315b, an imaging data 313a, 315c, a network adapter 318, a storage device 315, an input/output interface 317 and a display adapter 319. Each of remote computing devices 200, 300, 400 may further include a display device 330 and a data acquisition device 350. In embodiments, the system bus 312 may connect the subsystems to each other.

In embodiments, the computing device 310 and the remote computing devices 200, 300, 400 may be configured to perform one or more of the methods, functions, and/or operations presented herein. Computing devices that implement at least one or more of the methods, functions, and/or operations described herein may comprise an application or applications operating on at least one computing device. The computing device may comprise one or more computers and one or more databases. The computing device may be a single device, a distributed device, a cloud-based computer, or a combination thereof.

It shall be noted that the present disclosure may be implemented in any instruction-execution/computing device or system capable of processing data, including, without limitation laptop computers, desktop computers, and servers. The present disclosure may also be implemented into other computing devices and systems. Furthermore, aspects of the present disclosure may be implemented in a wide variety of ways including software (including firmware), hardware, or combinations thereof. For example, the functions to practice various aspects of the present disclosure may be performed by components that are implemented in a wide variety of ways including discrete logic components, one or more application specific integrated circuits (ASICs), and/or program-controlled processors. It shall be noted that the manner in which these items are implemented is not critical to the present disclosure.

Meanwhile, enzymes are essential proteins for living organisms that act as catalysts to accelerate biochemical reactions in the body. With over 4,000 enzymes responsible for chemical reactions in living organisms, it is necessary to organize the properties and functionalities of these enzymes, Enzyme Commission (EC) numbers have been assigned to enzymes to mark their functions, helping the process of inferring and analyzing biological and genetic properties of new species through the study of their protein sequences. Along with the findings of countless enzymes, there are currently over 565,928 manually curated protein sequences and over 225,013,025 that are computationally annotated by EC numbers. EC numbers are composed of four digits representing each of the following: class, sub-class, sub-subclass, and serial number.

Each of these digits is used to describe which reactions the enzymes act as catalysts. For, instance, the first digit which shows the class, identifies the enzyme as one of the following 7 groups, with Translocases being a relatively new class: Oxidoreductases (EC 1), Transferases (EC 2), Hydrolases (EC 3), Lyases (EC 4), Isomerases (EC 5) Ligases (EC 6) and Translocases (EC 7). The sub-class and sub-subclass further help classify the enzyme based on reactions, and the serial number is a unique number given to enzymes to identify them. This hierarchical classification system of enzymes allows for further analysis and inference of functions of genes and properties of genomes by looking at the protein sequences.

The present apparatus and method comprise three modules: preprocessing, feature extraction, and enzyme commission (EC) number prediction. The preprocessing module converts the amino acid sequence into a 2-D matrix embedding representation, preparing it for input into the convolutional neural network (CNN)-based feature extraction module. The CNN-based feature extraction module takes the 2-D matrix embedding as input and generates feature maps that mathematically represent the features of the input amino acid sequence. These extracted feature maps are then fed into the EC number prediction network. Each module will be explained in more detail below.

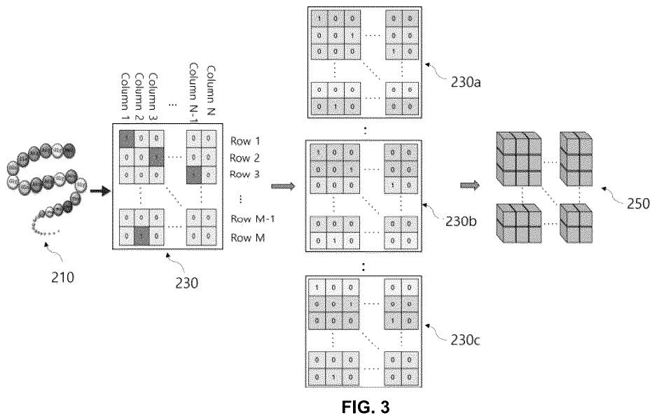

FIG. 3 is a view showing an illustrative process for preprocessing an amino acid sequence by an apparatus according to embodiments of the present disclosure.

As depicted, the preprocessing may include converting, creating and transforming. Firstly, the amino acid sequence 210 may be converted into an initial 2-dimensional embedding matrix 230 which has M rows and N columns by the processor 111, 311. In this case, the matrix may be composed of 1,000 rows for each individual amino acid in the sequence, and 21 columns with each column representing one of the 21 types of amino acids.

Secondly, the 2-dimensional embedding matrices 230a, 230b, 230c may be newly created in a plural using the initial 2-dimensional embedding matrix 230. For example, the plurality of the 2-dimensional embedding matrices 230a, 230b, 230c may be newly created by shifting at least one row of M rows in a column direction of the matrix. The shifting may be done in variable ways.

Thirdly, the plurality of the 2-dimensional embedding matrices 230a, 230b, 230c, . . . may be transformed into a 3-dimensional embedding matrix 250 by stacking the plurality of the 2-dimensional embedding matrices together. In this case, the stacking order is desirable in the order in which the 2-dimensional embedding matrices were created, but may be stacked randomly regardless of the order.

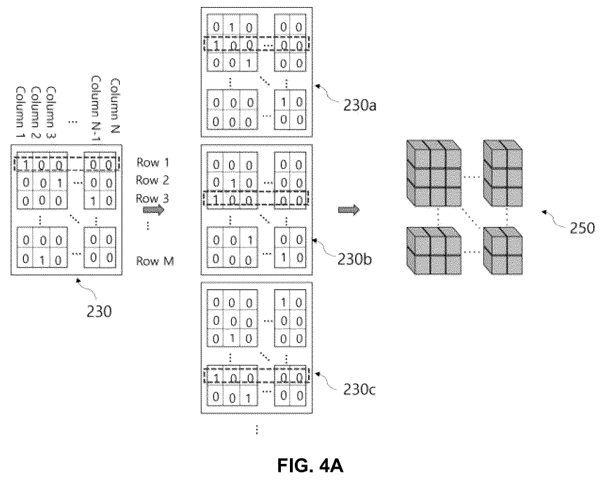

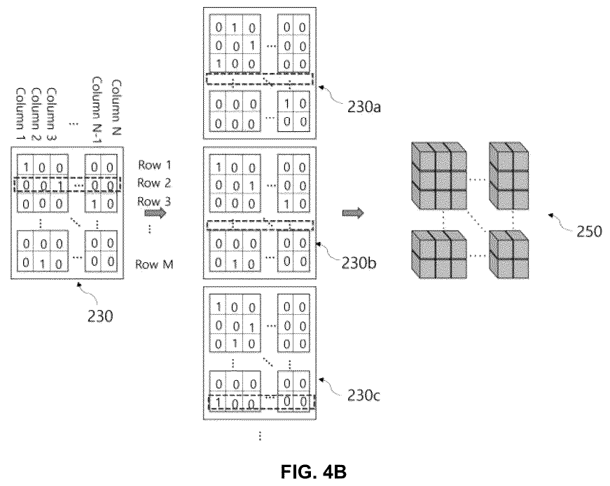

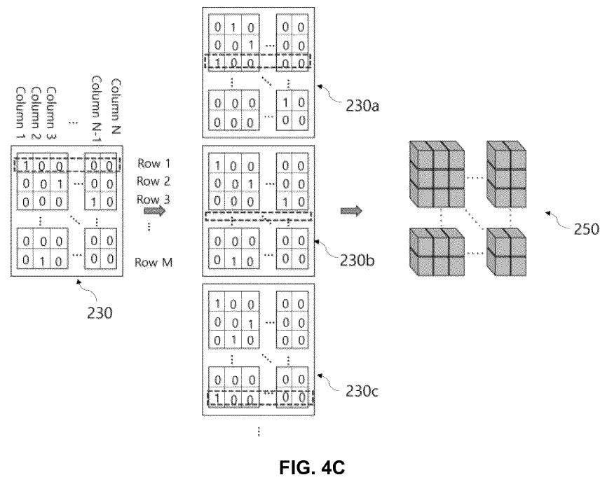

FIGS. 4A to 4C are an exemplary diagram showing a transformed 3-dimensional embedding matrix after various shifting of an initial 2-dimensional embedding matrix according to embodiments of the present disclosure.

As depicted in FIG. 4A, the plurality of 2-dimensional embedding matrices 230a, 230b, 230c, . . . may be created by sequentially shifting one row of the M rows in each column direction of the 2-dimensional embedding matrix. In this case, the shifting is moving one space in each column direction. The generated 2-dimensional embedding matrices 230a, 230b, 230c, . . . are stacked in the order in which the 2-dimensional embedding matrices were created, thereby transforming a 3-dimensional embedding matrix 250. As depicted in FIG. 4B, the plurality of 2-dimensional embedding matrices 230a, 230b, 230c, . . . may be created by sequentially shifting only even row of the M rows in each column direction of the 2-dimensional embedding matrix. In this case, the shifting means that the even row moves two spaces in a column direction. Also the generated 2-dimensional embedding matrices 230a, 230b, 230c are stacked in the order in which the 2-dimensional embedding matrices were created, thereby transforming a 3-dimensional embedding matrix 250. As depicted in FIG. 4C, the plurality of 2-dimensional embedding matrices 230a, 230b, 230c, may be created by sequentially shifting only odd row of the M rows in each column direction of the 2-dimensional embedding matrix. In this case, the shifting means that the odd row moves two spaces in a column direction. The generated 2-dimensional embedding matrices 230a, 230b, 230c, . . . are stacked in the order in which the 2-dimensional embedding matrices were created, thereby transforming a 3-dimensional embedding matrix 250. Although not depicted, the 2-dimensional embedding matrices 230a, 230b, 230c, . . . may be created by sequentially moving two or three rows in a column direction and in a variety of other ways.

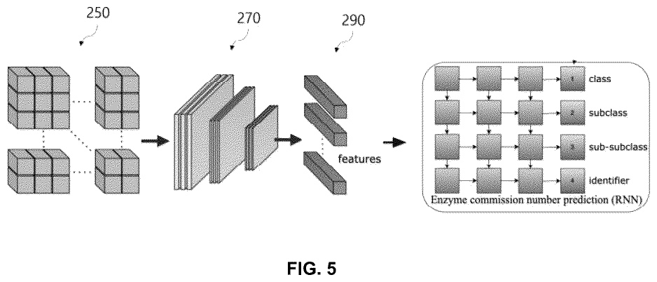

FIG. 5 is an exemplary diagram of an architecture of a machine learning model for learning a process according to embodiments of the present disclosure.

As depicted, the feature extractor 270 may exploit a machine learning model like a convolutional neural network designed to process the 3-dimensional embedding matrix 250. The 3-dimensional embedding matrix 250 may be a collection of 2-dimensional matrices stacked in various orders. The feature extractor 270 may extract a feature 290 of the amino acid sequence forming the 3-dimensional embedding matrix 250 and predict the enzyme commission number based on the extracted feature of the amino acid sequence.

The training of the feature extractor 270 may involve optimizing loss functions pertinent to the downstream tasks for predicting the enzyme commission number. Throughout the training phase, the feature extractor 270 may learn the ability to extract crucial features such as class, subclass, sub-subclass and identifier of the enzyme commission number.

In embodiments, the machine learning model 270 may be installed into a processor 111 and executed by the processor 111 in FIG. 1. The machine learning model 270 may be installed into a computer-readable medium or media (not shown in FIG. 1) and executed by the computer-readable medium or media. In alternative embodiments, the machine learning model 270 may be installed into the memory unit 113 or the storage device 115 and executed by the processor 111.

Meanwhile, for the training of the above network 270, a loss function may be utilized. In this case, the network 270 may include a recurrent neural network such as a long short-term model. The loss function may be the cross-entropy loss function for predicting the enzyme commission number. It is important to note that the feature extractor 270 is trained by the loss function due to the backpropagation algorithm's characteristics. The cross-entropy loss function may be employed for object classification tasks.

### Experiments

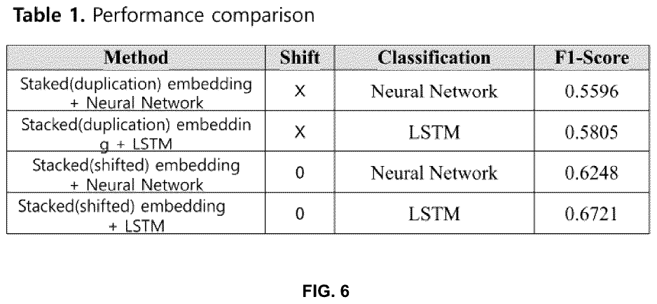

FIG. 6 shows a table of performance comparison of enzyme commission number prediction results achieved by the computing device shown in FIG. 1 based on comparison with 3-dimensional embedding matrix created by another method according to embodiments of the present disclosure.

The table 1 shown in FIG. 6 lists the performance comparison of the proposed method vs. stacked 3-dimensional embedding matrix by duplication method of 2-dimensional embedding matrix for the enzyme commission number prediction. For this comparative analysis, ResNet-101 is employed, all have demonstrated comparable performance in classification tasks.

The dataset was split such that 80% of the dataset was used to train the model and the remaining 20% was used for testing with a 5-fold cross-validation. This means that the dataset was split into 5 sets of samples and the training was done 5 times, rotating by which set will be used for testing. For each rotation, a varying number of K feature extractors were selected and the F1-score, which is the harmonic mean of the recall and precision, was calculated for each of the trained structures. For the feature extractors, it is experimented with a different number of LSTM layers for ResNet-101. On every rotation, 4 sets were used for training and the remaining 1 set was used to validate the trained model. The mean F1-score was then calculated along with the standard deviation and plotted along with the accuracy of the model.

As shown in table 1, in conclusion, the F1-score based on the proposed method is higher than stacked 3-dimensional embedding matrix by duplication method of 2-dimensional embedding matrix.

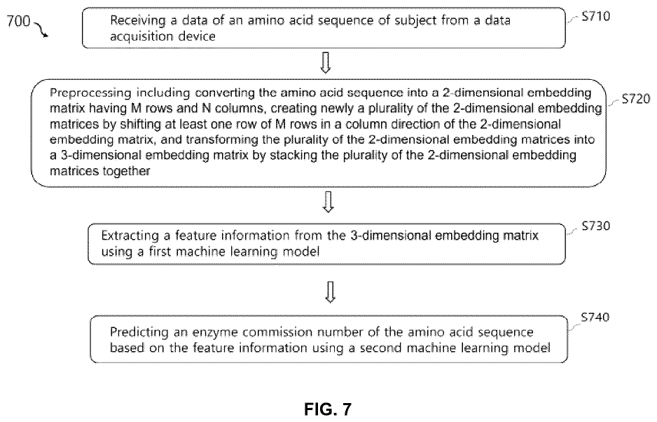

FIG. 7 is an exemplary flow diagram showing a predicting process of an enzyme commission number according to embodiments of present disclosure. The predicting process 700 may be performed by a suitable machine learning model like a convolutional neural network.

At step S710, the data of the amino acid sequence of plant subject is received from a data acquisition device. At step S720, the data of the amino acid sequence may be pre-processed by a processor. For example, the data of the amino acid sequence may be converted into a 2-dimensional embedding matrix having M rows and N columns, a plurality of the 2-dimensional embedding matrices may be created by shifting at least one row of M rows in a column direction of the 2-dimensional embedding matrix, and the plurality of the 2-dimensional embedding matrices may be transformed into a 3-dimensional embedding matrix by stacking the plurality of the 2-dimensional embedding matrices together.

At step S730, a first machine learning model may extract a feature information from the 3-dimensional embedding matrix. The feature information may include various information such as an amino acid sequence. At this time, the first machine learning model may transform the 3-dimensional embedding matrix into a feature map to visually encapsulate a specific feature in the 3-dimensional embedding matrix. At step S740, the second machine learning model may predict an enzyme commission number based on the feature information. In this case, the second machine learning model may employ a cross-entropy loss function in order to enhance the predicted value of the enzyme commission number.

Embodiments of the present disclosure may be encoded upon one or more non-transitory computer-readable media with instructions for one or more processors or processing units to cause steps to be performed. It shall be noted that the one or more non-transitory computer-readable media shall include volatile and non-volatile memory. It shall be noted that alternative implementations are possible, including a hardware implementation or a software/hardware implementation. Hardware-implemented functions may be realized using ASIC(s), programmable arrays, digital signal processing circuitry, or the like. Accordingly, the “means” terms in any claims are intended to cover both software and hardware implementations. Similarly, the term “computer-readable medium or media” as used herein includes software and/or hardware having a program of instructions embodied thereon, or a combination thereof. With these implementation alternatives in mind, it is to be understood that the figures and accompanying description provide the functional information one skilled in the art would require to write program code (i.e., software) and/or to fabricate circuits (i.e., hardware) to perform the processing required.

It shall be noted that embodiments of the present disclosure may further relate to computer products with a non-transitory, tangible computer-readable medium that have computer code thereon for performing various computer-implemented operations. The media and computer code may be those specially designed and constructed for the purposes of the present disclosure, or they may be of the kind known or available to those having skill in the relevant arts. Examples of tangible computer-readable media include, but are not limited to: magnetic media such as hard disks, floppy disks, and magnetic tape; optical media such as CD-ROMs and holographic devices; magneto-optical media; and hardware devices that are specially configured to store or to store and execute program code, such as application specific integrated circuits (ASICs), programmable logic devices (PLDs), flash memory devices, and ROM and RAM devices. Examples of computer code include machine code, such as produced by a compiler, and files containing higher level code that are executed by a computer using an interpreter. Embodiments of the present disclosure may be implemented in whole or in part as machine-executable instructions that may be in program modules that are executed by a processing device. Examples of program modules include libraries, programs, routines, objects, components, and data structures. In distributed computing environments, program modules may be physically located in settings that are local, remote, or both.

One skilled in the art will recognize no computing system or programming language is critical to the practice of the present disclosure. One skilled in the art will also recognize that a number of the elements described above may be physically and/or functionally separated into sub-modules or combined together.

It will be appreciated to those skilled in the art that the preceding examples and embodiment are exemplary and not limiting to the scope of the present invention. It is intended that all permutations, enhancements, equivalents, combinations, and improvements thereto that are apparent to those skilled in the art upon a reading of the specification and a study of the drawings are included within the true spirit and scope of the present invention.

What is claimed is:

1. An apparatus for predicting an enzyme commission number using an amino acid sequence of a subject, comprising:
a processor; and
a memory comprising one or more sequences of instructions which, when executed by the processor, causes steps to be performed comprising:
preprocessing including converting the amino acid sequence into a 2-dimensional embedding matrix having M rows and N columns, creating newly a plurality of the 2-dimensional embedding matrices by shifting at least one row of M rows in a column direction of the 2-dimensional embedding matrix, and transforming the plurality of the 2-dimensional embedding matrices into a 3-dimensional embedding matrix by stacking the plurality of the 2-dimensional embedding matrices together;
extracting a feature of the amino acid sequence from the 3-dimensional embedding matrix using a machine learning model; and
predicting the enzyme commission number based on the extracted feature of the amino acid sequence.

2. The apparatus of claim 1, wherein the plurality of the 2-dimensional embedding matrix is created by sequentially shifting one row of the M rows in the column direction.

3. The apparatus of claim 1, wherein the plurality of the 2-dimensional embedding matrix is created by sequentially shifting an even row of the M rows in the column direction.

4. The apparatus of claim 1, wherein the plurality of the 2-dimensional embedding matrix is created by sequentially shifting an odd row of the M rows in the column direction.

5. The apparatus of claim 1, wherein the machine learning model includes convolutional neural network or Long Short-Term Model.

6. A non-transitory computer-readable medium or media comprising one or more sequences of instructions which, when executed by a processor, causes steps for predicting an enzyme commission number using an amino acid sequence of a subject, comprising:
receiving data of the amino acid sequence
preprocessing including converting the data of the amino acid sequence into a 2-dimensional embedding matrix having M rows and N columns, creating newly a plurality of the 2-dimensional embedding matrices by shifting at least one row of M rows in a column direction of the 2-dimensional embedding matrix, and transforming the plurality of the 2-dimensional embedding matrices into a 3-dimensional embedding matrix by stacking the plurality of the 2-dimensional embedding matrices together;
extracting a feature of the amino acid sequence from the 3-dimensional embedding matrix using a machine learning model; and
predicting the enzyme commission number based on the extracted feature of the amino acid sequence.

7. The non-transitory computer-readable medium or media of claim 6, wherein the plurality of the 2-dimensional embedding matrix is created by sequentially shifting one row of the M rows in the column direction.

8. The non-transitory computer-readable medium or media of claim 6, wherein the plurality of the 2-dimensional embedding matrix is created by sequentially shifting an even row of the M rows in the column direction.

9. The non-transitory computer-readable medium or media of claim 6, wherein the plurality of the 2-dimensional embedding matrix is created by sequentially shifting an odd row of the M rows in the column direction.
